(19) United States
(12) Patent Application Publication
(10) Pub. No.: US 2023/0185100 A1
(43) Pub. Date: Jun. 15, 2023

(54) KALEIDOSCOPIC DISPLAY ASSEMBLIES

(71) Applicant: ALL PLASTIC, INC., Rancho Cordova, CA (US)

(72) Inventors: William Thomas SMITH, Fair Oaks, CA (US); Cheryl Ann SMITH, Fair Oaks, CA (US)

(21) Appl. No.: 18/081,423

(22) Filed: Dec. 14, 2022

Related U.S. Application Data
(60) Provisional application No. 63/289,839, filed on Dec. 15, 2021.
(60) Provisional application No. 63/325,059, filed on Mar. 29, 2022.

(51) Int. Cl. G02B 27/08; G01N 33/00; G01N 21/84; G01N 21/01

(52) U.S. Cl. CPC G02B 27/08; G01N 33/0098; G01N 21/84; G01N 21/01; G01N 33/0062; G01N 2021/8466; G01N 2021/0112

(57) ABSTRACT

A kaleidoscopic display assembly, comprising: (a) a stand; (b) a kaleidoscope tube supported by the stand; (c) a display container in alignment with the kaleidoscope tube for containing at least a first botanical specimen viewable in a kaleidoscopic pattern through a viewing opening of the kaleidoscope tube; and (d) an aroma container positioned alongside the tube and enclosing an aroma chamber for containing a second botanical specimen. The aroma container has one or more scent ports extending therethrough for sampling an aroma of the second botanical specimen during viewing of the kaleidoscopic pattern.

### CROSS-REFERENCE TO RELATED APPLICATIONS

This application claims the benefit of priority to U.S. Provisional Application No. 63/289,839, filed Dec. 15, 2021, and U.S. Provisional Application No. 63/325,059, filed Mar. 29, 2022, each of which is hereby incorporated herein by reference in its entirety.

### FIELD

The specification relates generally to display of articles, and more specifically, to kaleidoscopic display assemblies.

### BACKGROUND

Kaleidoscopes are optical instruments with two or more reflective surfaces angled relative to each other for producing symmetrical patterns of objects located at one end of the reflective surfaces due to repeated reflection. The symmetrical patterns are viewable from the other end of the reflective surfaces.

### SUMMARY

The following summary is intended to introduce the reader to various aspects of the applicant's teaching, but not to define any invention.

According to some aspects, a kaleidoscopic display assembly includes: (a) a stand; (b) a kaleidoscope tube supported by the stand and extending along a tube axis between a first end and a second end. The tube has an interior extending along the tube axis between a first opening at the first end of the tube and a second opening at the second end of the tube, and a plurality of reflective surfaces arranged in the interior about the tube axis for forming kaleidoscopic patterns. The display assembly further includes (c) a display container at the second end of the tube. The display container encloses a first display chamber extending across the second opening of the tube for containing at least a first botanical specimen viewable in a kaleidoscopic pattern through the first opening of the tube. The display assembly further includes (d) an aroma container alongside the tube and enclosing an aroma chamber for containing a second botanical specimen. The aroma container has one or more scent ports extending therethrough for sampling an aroma of the second botanical specimen during viewing of the kaleidoscopic pattern.

In some examples, the aroma container includes an air pump operable to discharge scented air having an aroma of the second botanical sample from the aroma chamber through the one or more scent ports.

In some examples, the air pump comprises a resiliently deformable bulb projecting outwardly from a wall of the aroma container, and depressible inwardly toward the aroma chamber to urge discharge of the scented air.

In some examples, the display container includes a second display chamber extending across the second opening. The second display chamber contains a plurality of objects for forming part of the kaleidoscopic pattern viewable through the first opening.

In some examples, the display container is rotatable relative to the kaleidoscope tube about a display container axis extending parallel with the tube axis for moving the first botanical specimen relative to the reflective surfaces.

In some examples, the display container includes a container body and a removable lid attached to the container body. The container body and the lid enclose the first display chamber.

In some examples, the lid has a second display chamber containing a plurality of objects for forming part of the kaleidoscopic pattern viewable through the first opening.

In some examples, the stand includes a base and a frame extending upwardly from the base, and the kaleidoscope tube, aroma container, and display container are coupled to the frame.

In some examples, the display assembly includes a lighting system having at least one light source coupled to the stand for illuminating contents of the display container.

According to some aspects, a kaleidoscopic display assembly includes: (a) a stand; (b) a kaleidoscope tube supported by the stand; (c) a display container in alignment with the kaleidoscope tube for containing at least a first botanical specimen viewable in a kaleidoscopic pattern through a viewing opening of the kaleidoscope tube; and (d) an aroma container adjacent the tube and enclosing an aroma chamber for containing a second botanical specimen. The aroma container has one or more scent ports extending therethrough for sampling an aroma of the second botanical specimen during viewing of the kaleidoscopic pattern.

In some examples, the aroma container includes an air pump operable to discharge scented air having an aroma of the second botanical sample from the aroma chamber through the one or more scent ports.

In some examples, the display container includes a first display chamber for containing the first botanical specimen and a second display chamber in isolation from the first display chamber and containing a plurality of objects for forming part of the kaleidoscopic pattern.

According to some aspects, a kaleidoscopic display assembly includes: (a) a stand; (b) a kaleidoscope tube supported by the stand; (c) one or more containers coupled to the stand and enclosing one or more corresponding chambers for containing corresponding botanical specimens. One of the chambers is in alignment with the kaleidoscope tube for forming a kaleidoscopic pattern of a corresponding botanical specimen in the at least one of the chambers. The kaleidoscopic pattern is viewable through a viewing opening of the kaleidoscope tube. One of the containers includes one or more scent ports for sampling an aroma of a corresponding botanical specimen during viewing of the kaleidoscopic pattern.

In some examples, the one of the containers includes an air pump operable to urge discharge of scented air having an aroma of the corresponding botanical specimen through the one or more scent ports.

According to some aspects, a method of displaying botanical specimens for multisensory sampling includes: (a) inserting a first botanical specimen into a display chamber in alignment with a kaleidoscope tube for forming a kaleidoscopic pattern of the first botanical specimen viewable through the kaleidoscope tube; and (b) inserting a second botanical specimen into an aroma container mounted adjacent the kaleidoscope tube. The aroma container has one or more scent ports for sampling an aroma of the second botanical specimen during viewing of kaleidoscopic pattern.

In some examples, the first botanical specimen and the second botanical specimen are of a common strain.

According to some aspects, a kaleidoscopic display assembly includes: (a) a stand; (b) a kaleidoscope tube supported by the stand, the tube having an interior extending along a tube axis between a first opening and a second opening opposite the first opening, and a plurality of reflective surfaces arranged in the interior about the tube axis for forming kaleidoscopic patterns; (c) a first display chamber extending across the second opening for containing a botanical specimen viewable in a kaleidoscopic pattern through the first opening; and (d) a second display chamber extending across the second opening axially intermediate the first display chamber and the second opening of the kaleidoscope tube. The second display chamber containing a plurality of objects for forming part of the kaleidoscopic pattern viewable through the first opening.

In some examples, the first display chamber and the second display chamber are in a common display container coupled to the stand. In some examples, the display container has a container body and a lid removably attached to the container body. The container body and the lid bound the first display chamber, and the second chamber is internal the lid.

In some examples, each of the first and second display chambers are rotatable relative to the kaleidoscope tube for moving the botanical specimen and the plurality of objects relative to the reflective surfaces.

### BRIEF DESCRIPTION OF THE DRAWINGS

The drawings included herewith are for illustrating various examples of apparatuses, articles, and methods of the present specification and are not intended to limit the scope of what is taught in any way. In the drawings:

FIG. 1 is a perspective view of an example kaleidoscopic display assembly;

FIG. 2 is a rear elevation view of the display assembly of FIG. 1

FIG. 3 is a cross-sectional view of the display assembly of FIG. 1; taken along line 3-3 of FIG. 2;

FIG. 4 is a cross-sectional view of a kaleidoscope tube portion of the display assembly of FIG. 1, taken along line 4-4 in FIG. 3;

FIG. 5 is an enlarged view of a display container portion of FIG. 3;

FIG. 6 is an enlarged view of an aroma container portion of FIG. 3, showing an air pump of the display assembly in an expanded state;

FIG. 7 is an enlarged view like that of FIG. 6, but showing the air pump in a contracted state;

FIG. 8 is a partially exploded perspective view of the display assembly of FIG. 1;

FIG. 9 is a perspective view of another example kaleidoscopic display assembly;

FIG. 10 is a partially exploded perspective view of a portion of the display assembly of FIG. 9;

FIG. 11 is a another partially exploded perspective view of a portion of the display assembly of FIG. 9; and

FIG. 12 is a front view of a portion of the display assembly of FIG. 9.

### DETAILED DESCRIPTION

Various apparatuses, articles, or processes will be described below to provide an example of an embodiment of each claimed invention. No embodiment described below limits any claimed invention and any claimed invention may cover apparatuses, articles, or processes that differ from those described below. The claimed inventions are not limited to apparatuses, articles, or processes having all of the features of any one apparatus, article, or process described below or to features common to multiple or all of the apparatuses, articles, or processes described below. It is possible that an apparatus, article, or process described below is not an embodiment of any claimed invention. Any invention disclosed in an apparatus, article, or process described below that is not claimed in this document may be the subject matter of another protective instrument, for example, a continuing patent application, and the applicants, inventors, or owners do not intend to abandon, disclaim, or dedicate to the public any such invention by its disclosure in this document.

In retail or other environments, product specimens are often put on display for viewing by potential consumers. In some cases, it may be desirable to display the product specimens in a unique manner to attract potential consumers to the product, and/or to allow potential consumers to also sample an aroma of certain product specimens (where the aroma may be a consideration in the selection and/or purchase of the product). The present application discloses aspects of display assemblies that can produce kaleidoscopic patterns of product specimens for viewing by potential consumers, and in some examples, also facilitate aroma sampling of the product specimens during viewing of the kaleidoscopic patterns. The product specimens can include, for example, botanical specimens such as, for example, cannabis flowers (commonly referred to as “buds”).

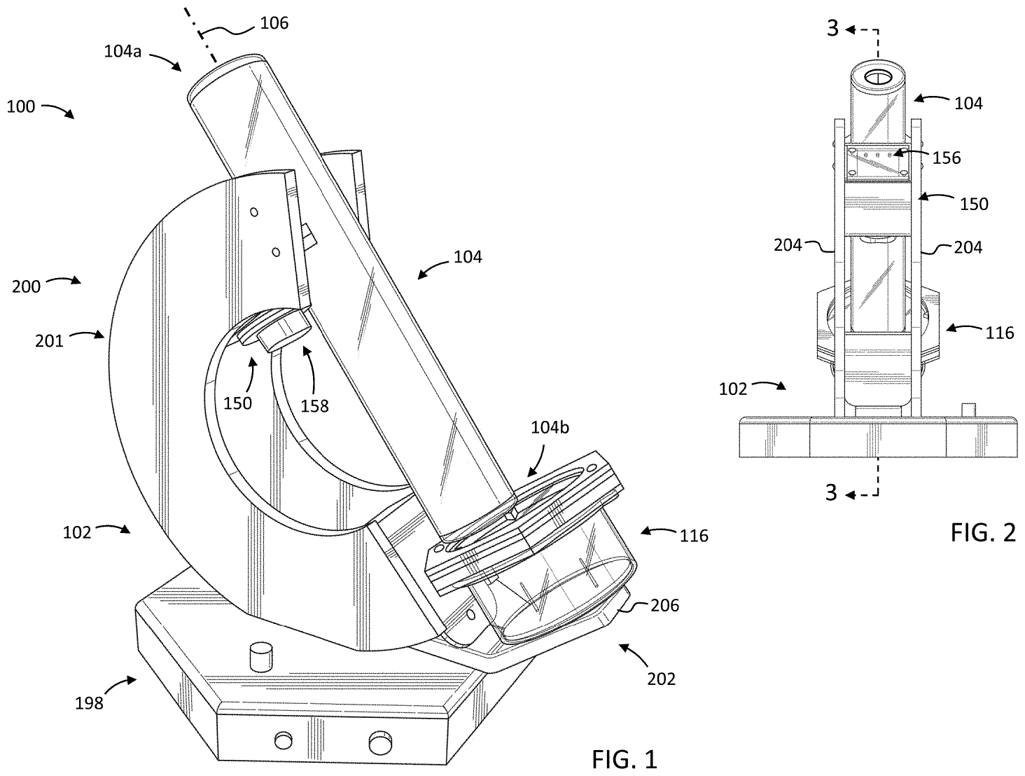

Referring to FIG. 1, an example kaleidoscopic display assembly 100 is illustrated. The display assembly 100 includes a stand 102 and a kaleidoscope tube 104 supported by the stand 102. In the example illustrated, the kaleidoscope tube 104 extends along a tube axis 106 between a first end 104a and a second end 104b opposite the first end 104a.

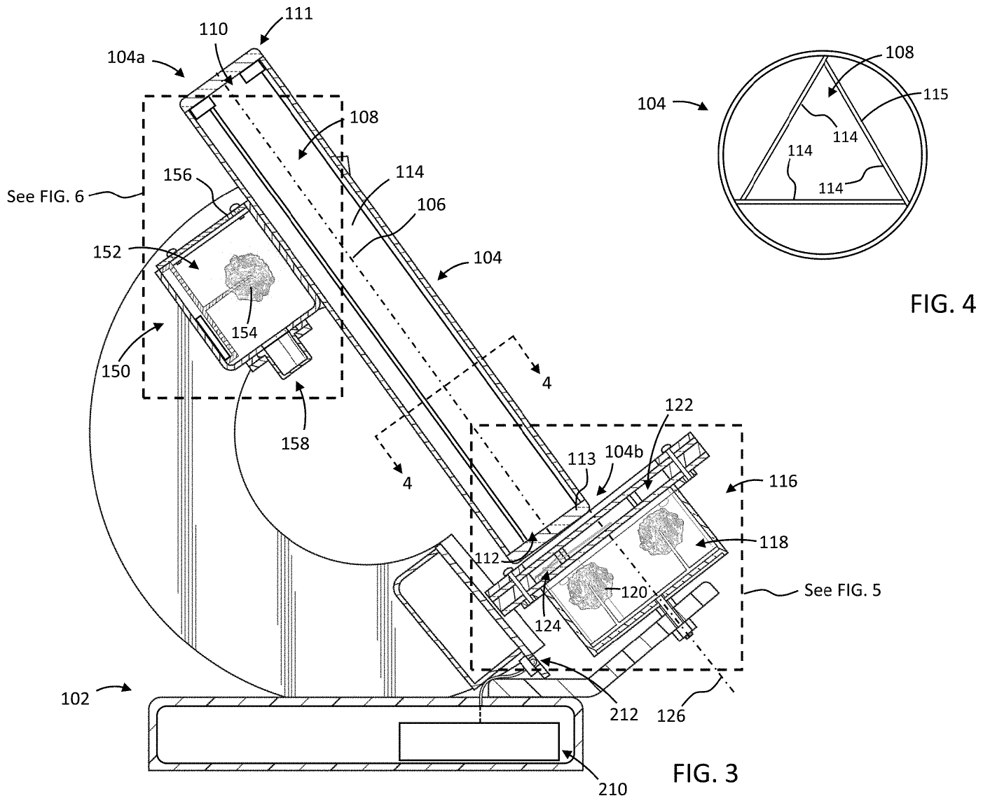

Referring to FIG. 3, in the example illustrated, the tube 104 has an interior 108 extending along the tube axis 106 between a first opening 110 (also referred to as a viewing opening) at the first end 104a of the tube 104 and a second opening 112 at the second end 104b of the tube 104. In the example illustrated, the tube axis 106 is oriented at an incline from horizontal, with the first end 104a of the tube 104 at an elevation above the second end 104, and with the first opening 110 angled upwardly and the second opening 112 angled downwardly. In the example illustrated, the tube 104 has an eyepiece 111 extending across the first opening 110 and a transparent endcap 113 extending across the second opening 112. The eyepiece 111 and endcap 113 generally seal the interior 108.

Referring to FIGS. 3 and 4, in the example illustrated, a plurality of reflective surfaces 114 are arranged in the interior 108 about the tube axis 106 (and angled relative to each other) for forming kaleidoscopic patterns of objects in front of the second opening 112. The kaleidoscopic patterns are viewable through the first opening 110. In the example illustrated, the reflective surfaces 114 comprise a plurality of generally planar mirrors 115 (three, in the example illustrated).

In the example illustrated, the tube 104 has a tubular housing having a generally circular cross section and in which the mirrors 115 are positioned. In other examples, the tubular housing can have a different cross-sectional shape (e.g. triangular, square, polygonal, etc.), or can be omitted, in which case the mirrors 115 (or other substrate with inwardly directed reflective surfaces) can define the interior and the first and second openings of the kaleidoscope tube.

Referring to FIG. 3, in the example illustrated, a display container 116 is positioned at the second end 104b of the kaleidoscope tube 104. The display container 116 encloses a first display chamber 118 containing at least a first botanical specimen 120. The first display chamber 118 extends across the second opening 112 (and intersects the tube axis 106) to form a kaleidoscopic pattern based on the first botanical specimen 120 for viewing through the first opening 110.

In the example illustrated, the display container 116 further includes a second display chamber 122 extending across the second opening 112 (and intersecting the tube axis 106). The second display chamber 122 contains a plurality of objects 124 for forming part of the kaleidoscopic pattern viewable through the first opening 110. In the example illustrated, the second display chamber 122 is positioned between the first display chamber 118 and the second opening 112 of the kaleidoscope tube 104, and the objects 124 serve as foreground to the first botanical specimen 120 in the kaleidoscopic pattern. In the example illustrated, the objects 124 can have optical properties suitable for producing kaleidoscopic effects. The objects can be, for example, of different colors relative to each other, transparent, and/or reflective for creating kaleidoscopic effects. The objects 124 can comprise, for example, material fragments such as colored pieces of glasses and/or other transparent (and/or opaque) materials for reflection into the kaleidoscopic pattern.

In the example illustrated, the display container 116 (including both the first and second chambers 118, 122) is rotatably mounted to the stand 102, and rotatable relative to the kaleidoscope tube 104 about a display container axis 126 for moving the contents of the display container 116 (e.g. the botanical specimen 120 and the objects 124 in the example illustrated) relative to the reflective surfaces 114. In the example illustrated, the display container axis 126 extends generally parallel with the tube axis 106. In the example illustrated, the display container axis 126 is spaced apart (offset) from the tube axis 106. In the example illustrated, the display container axis 126 extends through the center of the display container 116.

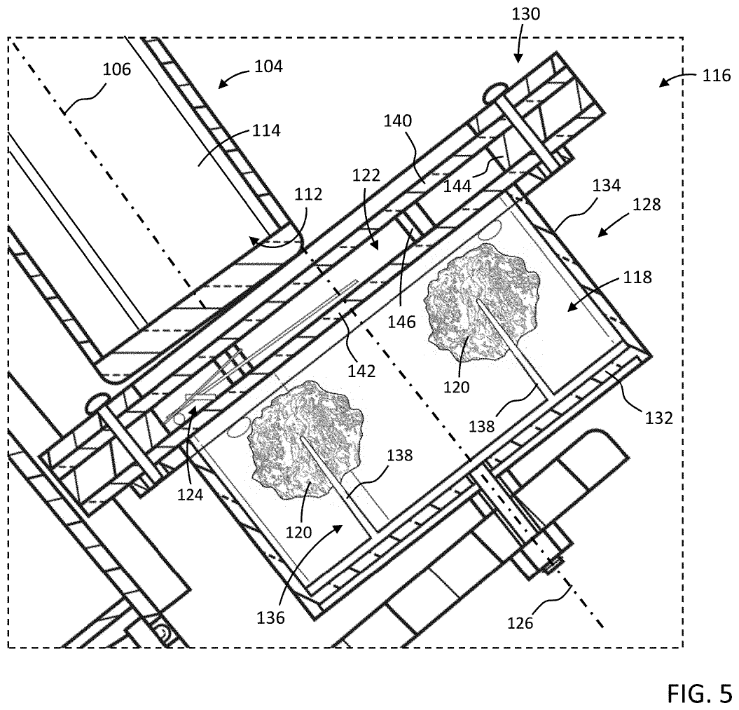

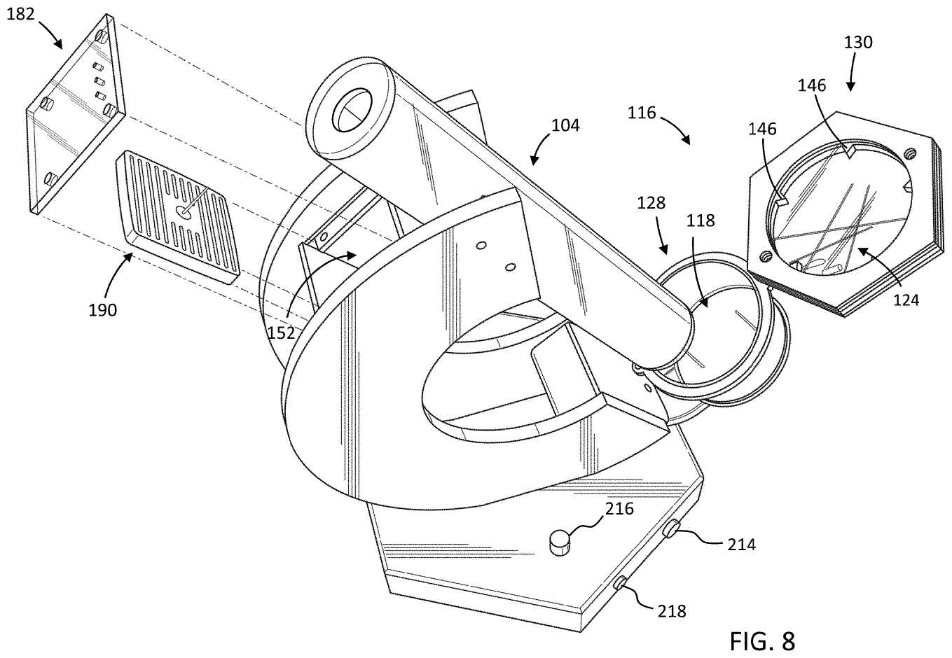

Referring to FIG. 5, in the example illustrated, the display container 116 has a display container body 128 and a display container lid 130 attached to the container body 128. The container body 128 and the lid 130 enclose the first display chamber 118. In the example illustrated, the container body 128 and at least a portion of the lid 130 are generally transparent to allow for illumination of the first and second display chambers 118, 122 (and the botanical specimens and objects therein). Referring to FIG. 8, in the example illustrated, the lid 130 is removable from the container body 128 for accessing the first display chamber 118 (e.g. for insertion and/or removal of the botanical specimen 120), and the lid 130 is securable to the container body 128 (e.g. through one or more fasteners) to inhibit unauthorized access to the first display chamber 118.

Referring to FIG. 5, in the example illustrated, the lid 130 is positioned between the container body 128 and the second opening 112 of the kaleidoscope tube 104, and is oriented generally normal to the container axis 126. At least the portion of the lid 130 extending across the second opening 112 is transparent to permit viewing of the botanical specimen 120 therethrough.

In the example illustrated, the container body 128 includes a container endwall 132 oriented normal to the container axis 126, and a container sidewall 134 extending axially along the container axis 126 from a periphery of the container endwall 132 to the lid 130. In the example illustrated, the first display chamber 118 is bounded axially by the lid 130 and the container endwall 132. The first display chamber 118 is bounded radially by the container sidewall 134. In the example illustrated, the first display chamber 118 is generally cylindrical.

The display container 116 can optionally include one or more specimen retainers 136 in the first display chamber 118 for retaining corresponding first botanical specimens 120 at one or more corresponding predetermined locations in the first display chamber 118. In the example illustrated, the display container 116 includes a plurality of the specimen retainers 136 in the first display chamber 118. In the example illustrated, the container 116 is rotatable about the container axis 126 relative to the reflective surfaces 114 to move the specimen retainers 136 into and out of alignment with the second opening 112 of the kaleidoscope tube 104 for viewing of the botanical specimen retained thereon. In the example illustrated, each specimen retainer 136 comprises at least one protrusion 138 projecting into the first display chamber 118 for engagement with the botanical specimen 120. In the example illustrated, each protrusion 138 projects from the endwall 132 into the first display chamber 118 generally parallel with the display container axis 126. In the example illustrated, each protrusion 138 is spaced radially apart from the display container axis 126, and the protrusions 138 are spaced generally equally apart from each other about the display container axis 126 (i.e. at 180 degrees apart from each other in the illustrated example with two protrusions). In the example illustrated, each protrusion 138 comprises a tapered mounting spike for impaling a corresponding botanical specimen 120 thereon.

In the example illustrated, the second display chamber 122 is contained in the container lid 130. In the example illustrated, the lid 130 has a pair of transparent layers 140, 142 spaced axially apart from each other, and the second display chamber 122 is between (and bounded axially by) the transparent layers 140, 142. The lid 130 has a lid sidewall 144 extending axially between the transparent layers 140, 142. In the example illustrated, the second display chamber 122 is bounded radially by the lid sidewall 144. In the example illustrated, the second display chamber 122 is generally cylindrical. In the example illustrated, a plurality of projections 146 are spaced circumferentially apart about the display container axis 126 and protrude radially inwardly into the second display chamber 122 (from the lid sidewall 144 in the example illustrated) to help disperse and randomize movement of the objects 124 in the second chamber 122 during rotation of the display container 116.

Referring to FIG. 3, in the example illustrated, the display assembly 100 further includes an aroma container 150 enclosing an aroma chamber 152 for containing a second botanical specimen 154. In the example illustrated, the aroma container 150 is separate and spaced apart from the display container 116. The aroma container 150 has one or more scent ports 156 extending through a wall of the container 150 between the aroma chamber 152 and an outside environment for sampling an aroma of the second botanical specimen 154 during viewing of the kaleidoscopic pattern through the first opening 110. The first and second botanical specimens 120, 154 can be of a common strain to facilitate multisensory (visual and olfactory) sampling of the same type of botanical specimen. In the example illustrated, the aroma container 150 is positioned alongside the kaleidoscope tube 104. In the example illustrated, the aroma container 150 is fixed to the stand 102 adjacent an underside of the kaleidoscope tube 104.

In the example illustrated, the aroma container 150 comprises a plurality of the scent ports 156. The scent ports 156 are directed axially along the tube axis 106 generally toward the first opening 110 of the kaleidoscope tube 104 for directing scented air discharged from the aroma chamber 152 toward a nose of a user viewing the kaleidoscopic pattern through the first opening 110. The scent ports 156 are sized to inhibit unauthorized access into the aroma chamber through the scent ports 156 while permitting discharge of scented air from the aroma chamber 152. In the example illustrated, each scent port 156 has a diameter of less than ⅛ inch (3.2 mm). In some examples, the diameter of each port 156 can be between 1/32 inch (0.8 mm) and 3/32 inch (2.4 mm). In some examples, the diameter of each scent port 156 can be approximately 1/16 inch (1.6 mm).

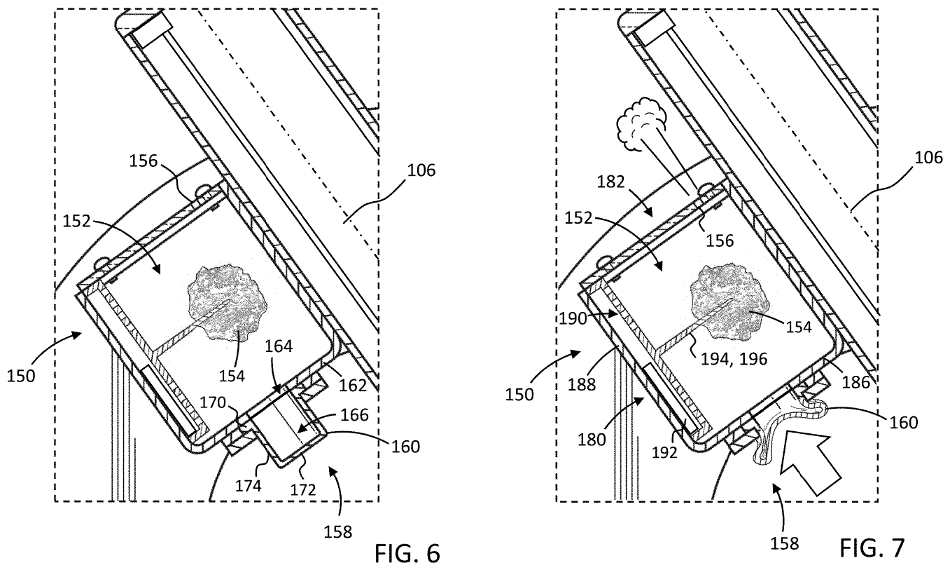

In the example illustrated, the aroma container 150 has an integrated air pump 158 operable to urge discharge of scented air having the aroma of the second botanical specimen 154 from the aroma chamber 152 through the scent ports 156. In the example illustrated, the air pump 158 cooperates with the aroma chamber 152 to define an interior volume of the aroma container 150. The air pump 158 is movable between an expanded state (shown in FIGS. 3 and 6) and a contracted state (shown in FIG. 7). Movement of the air pump 158 toward the contracted state reduces the interior volume for discharging the scented air from the aroma chamber 152 to the environment through the scent ports 156. Movement of the air pump 158 toward the expanded state increases the interior volume for drawing air into the interior volume from the environment through the scent ports 156.

Referring to FIG. 6, in the example illustrated, the air pump 158 comprises a resiliently deformable bulb 160. In the example illustrated, the bulb 160 is mounted over an aperture 164 in a wall 162 of the container 150, and an interior air chamber 166 in the bulb 160 is in fluid communication with the aroma chamber 152 via the aperture 164 to define the interior volume. When the air pump 158 is in the expanded state, the bulb 160 projects outwardly from the wall 162 of the aroma container 150 and is depressible inwardly toward the wall 162 (and the aroma chamber 152) to move the air pump 158 toward the contracted state and collapse the air chamber 166 for urging discharge of the scented air. After being depressed (see FIG. 7), the bulb 160 is urged outwardly away from the wall 162 to move the air pump 158 toward the expanded state and expand the air chamber 166. In the example illustrated, the bulb 160 is formed of an elastomeric material, and is of integral, unitary, one-piece construction. The elastomeric material has sufficient resiliency and is shaped to urge the air pump 158 back toward the expanded state when the bulb 160 is depressed and released.

In the example illustrated, the bulb 160 has a bulb base 170 mounted against the wall 162 about the aperture 164, a bulb endwall 172 opposite the bulb base 170 and spaced outwardly apart from the wall 162, and a bulb sidewall 174 extending between the bulb base 170 and the bulb endwall 172. The air chamber 166 in the bulb 160 is bounded by the bulb endwall 172 and the bulb sidewall 174. The air chamber 166 can be collapsed by pushing the endwall 172 toward the wall 162 (and aroma chamber 152) and/or squeezing together opposing sides of the bulb sidewall 174.

Referring to FIG. 7, in the example illustrated, the aroma container 150 has an aroma container body 180 and an aroma container lid 182 attached to the container body 180. The container body 180 and the lid 182 enclose the aroma chamber 152. In the example illustrated, the lid 182 is positioned axially intermediate (along the tube axis 106) the container body 180 and the first opening 110 of the kaleidoscope tube 104. In the example illustrated, the lid 182 is removable from the container body 180 for accessing the aroma chamber 152 (e.g. for insertion and/or removal of the second botanical specimen 154), and securable to the container body 180 (e.g. through one or more fasteners) to inhibit unauthorized access to the aroma chamber 152. In the example illustrated, the air pump 158 is mounted to the container body 180, with the deformable bulb 160 projecting axially from an underside surface of the container body 180. The scent ports 156 comprise a plurality of apertures passing through the lid 182 between the aroma chamber 152 and environment. In the example illustrated, the container body 180 has a container endwall 186 opposite the lid 182 (and oriented generally normal relative to the tube axis 106), and a container sidewall 188 extending axially (generally parallel with the tube axis 106) from a periphery of the container endwall 186 to the lid 182. In the example illustrated, the air pump 158 is mounted to the endwall 186 and projects axially therefrom toward the second opening 112.

In the example illustrated, the aroma container 150 has a vented partition 190 separating the aroma chamber 152 into a first compartment in which the botanical specimen is positioned and a second compartment in which a humidity control package 192 is positioned. The partition 190 has a plurality of vents for providing fluid communication between the first and second compartments to facilitate regulation of humidity in the aroma chamber 152.

The aroma container 150 can optionally include at least one specimen retainer 194 in the aroma chamber 152 for retaining the second botanical specimen 154 at a predetermined location in the aroma chamber 152. In the example illustrated, the specimen retainer 194 comprises at least one protrusion 196 projecting into the aroma chamber 152 for engagement with the second botanical specimen 154. In the example illustrated, the protrusion 196 projects into the aroma chamber 152 generally perpendicular to the tube axis 106. In the example illustrated, the protrusion 196 comprises a tapered mounting spike for impaling the botanical specimen 154 thereon. In the example illustrated, the specimen retainer 194 is fixed to and projects inwardly from the vented partition 190 toward a center of the aroma chamber 152.

In the example illustrated, the stand 102 has a base 198 and a frame 200 extending upwardly from the base 198. In the example illustrated, the frame 200 supports the kaleidoscope tube 104, display container 116, and aroma container 150 generally above the base 198. In the example illustrated, the frame 200 has a frame first portion 201 to which the kaleidoscope tube 104 and aroma container 150 are mounted, and a frame second portion 202 to which the display container 116 is mounted. In the example illustrated, the display container 116 is rotatably mounted to the frame second portion 202 for rotation about the display container axis 126.

Referring to FIG. 2, in the example illustrated, the frame first portion 201 includes a pair of upright members 204 (in the form of vertically oriented plates) spaced laterally apart from each other by a spacing. In the example illustrated, the kaleidoscope tube 104 and aroma container 150 are positioned in the spacing between the upright members 204 and fixed to the upright members 204. Referring to FIG. 1, in the example illustrated, the frame second portion 202 comprises a beam member 206 (in the form of a second plate oriented generally normal to the tube axis 106) rotatably supporting the display container 116 thereatop.

Referring to FIG. 3, in the example illustrated, the display assembly 100 includes a lighting system 210 having at least one light source 212 for illuminating contents of the display container 116 (e.g. the botanical specimen 120 in the first display chamber 118 and the objects 124 in the second display chamber 122). In the example illustrated, the light source comprises a light emitting diode (LED) coupled to the stand 102 adjacent the transparent display container body 128 for illuminating the first display chamber 118. In the example illustrated, the light source 212 is positioned alongside and directed toward the transparent sidewall of the display container 116. Referring to FIG. 8, in the example illustrated, the lighting system 210 further includes a power and charging port 214 for connection of the lighting system 210 to a power source for powering the lighting system 210 and/or charging a power storage unit (e.g. one or more batteries) for powering the lighting system 210. The lighting system 210 can further include a switch 216 for selectively activating, deactivating, and/or adjusting intensity (e.g. dimming) the light source 212, and an indicator light 218 for indicating, for example, power and/or charging status.

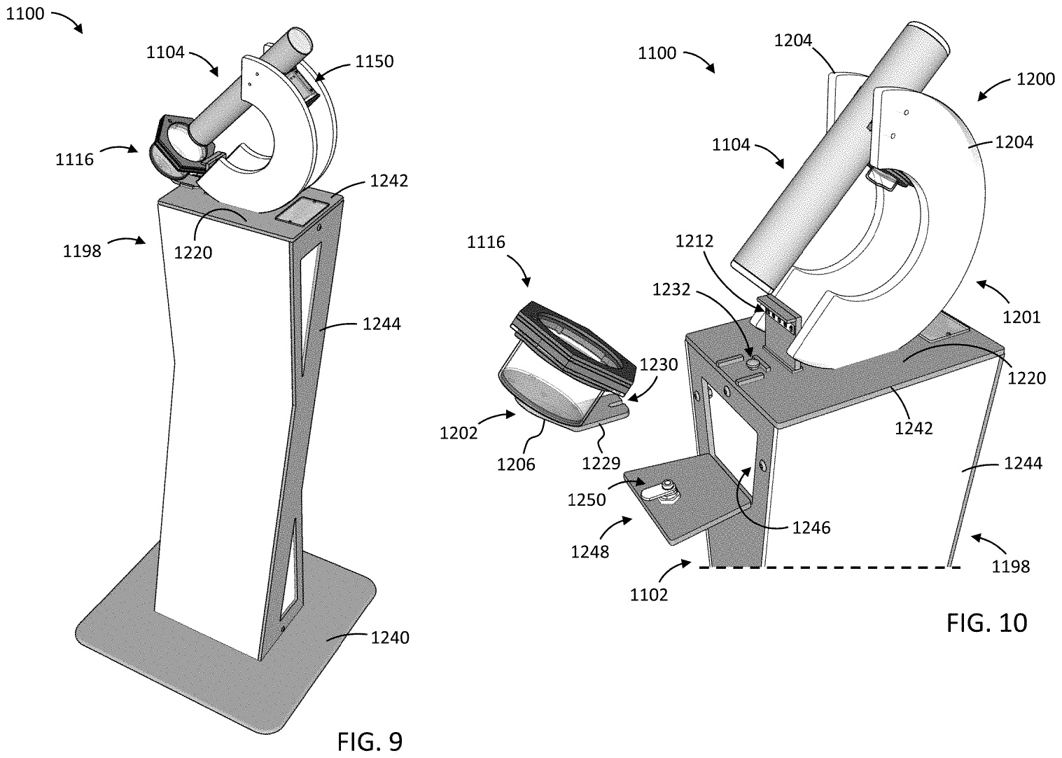

Referring to FIG. 9, another example kaleidoscopic display assembly 1100 is illustrated. The display assembly 1100 has similarities to the display assembly 100 and like features are identified with like reference characters, incremented by 1000.

In the example illustrated, the display assembly 1100 includes a stand 1102, a kaleidoscope tube 1104 supported by the stand 1102, and a display container 1116 positioned at one end of the kaleidoscope tube 1104. The display container 1116 is for containing a first botanical specimen and a plurality of kaleidoscopic objects viewable through the kaleidoscope tube 1104 as part of a kaleidoscopic pattern. In the example illustrated, the display assembly 1100 further includes an aroma container 1150 for sampling an aroma of a second botanical specimen contained in the aroma container 1150 during viewing of the kaleidoscopic pattern.

Referring to FIG. 10, in the example illustrated, the stand 1102 has a base 1198 and a frame 1200 supported by the base 1198. In the example illustrated, the frame 1200 supports the kaleidoscope tube 1104, display container 1116, and aroma container 1150 generally above the base 1198. In the example illustrated, the frame 1200 has a frame first portion 1201 to which the kaleidoscope tube 1104 and aroma container 1150 are mounted, and a frame second portion 1202 to which the display container 1116 is mounted. In the example illustrated, the display container 1116 is rotatably mounted to the frame second portion 1202.

In the example illustrated, the frame first portion 1201 includes a pair of upright members 1204 spaced laterally apart from each other by a spacing. In the example illustrated, each upright member 1204 comprises a vertically oriented plate (which is C-shaped, in the example illustrated). In the example illustrated, the kaleidoscope tube 1104 and aroma container 1150 are positioned in the spacing between the upright members 1204 and fixed thereto.

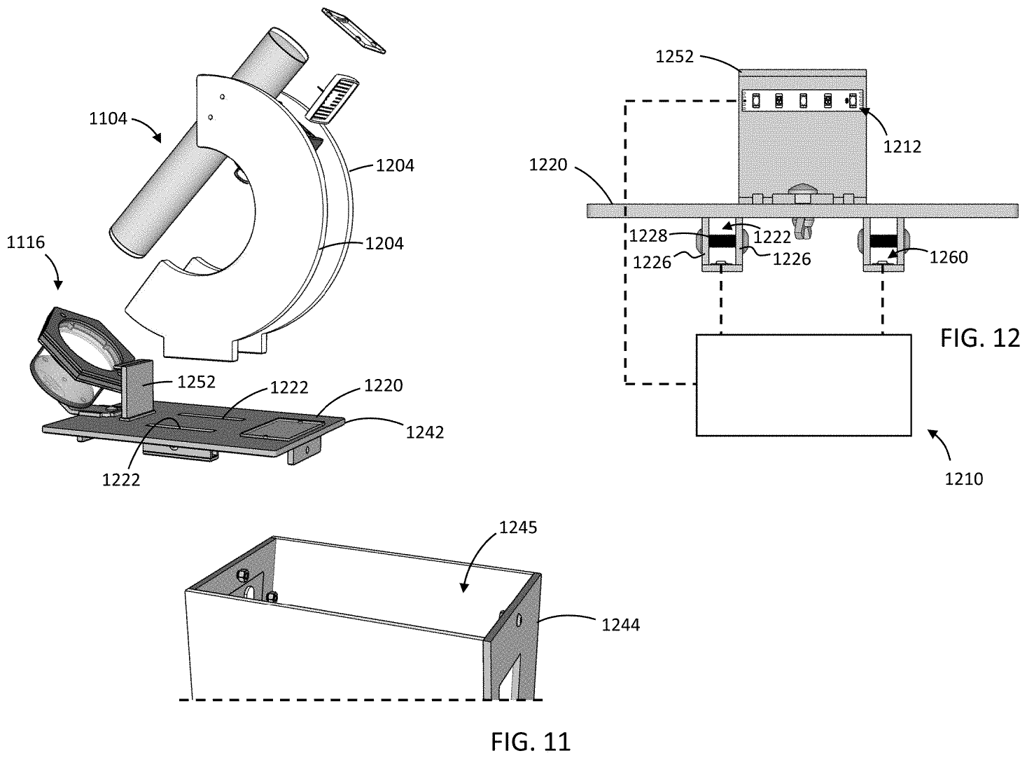

Referring to FIG. 11, in the example illustrated, the base 1198 of the pedestal has a top surface 1220 and a pair of laterally spaced apart mounting recesses 1222 in the top surface 1220. In the example illustrated, the recesses 1222 comprise elongated channels extending lengthwise between a front and rear of the display assembly 1100. Each upright member 1204 has a mounting portion 1224 at a lower end thereof and received in close fit in a corresponding recess 1222 for retaining the upright member 1204 in an upright position atop the base 1198. Referring to FIG. 12, in the example illustrate, each recess 1222 is defined by a pair of laterally spaced apart recess sidewalls 1226. At least one compression screw 1228 extends across each recess 1222 between the recess sidewalls 1226 for drawing the sidewalls 1226 together to exert a compressive force on the mounting portion 1224 when received in the recess 1222 to secure the upright member 1204 to the base 1198.

Referring to FIG. 10, in the example illustrated, the frame second portion 1202 includes a beam member 1206 comprising a plate oriented generally normal to the tube 1104 and rotatably supporting the display container 1116 thereatop. In the example illustrated, the frame second portion 1202 is detachably mounted to the base 1198. In the example illustrated, the beam member 1206 further includes a bracket portion 1229 extending rearwardly from the plate of the beam member 1206. The bracket portion 1229 has a mounting slot 1230 with an open end for sliding over a bracket fastener 1232 extending upwardly from the top surface 1220 of the base 1198 for securing the frame second portion 1202 to the base 1198.

Referring to FIG. 9, in the example illustrated, the base 1198 is in the form of a pedestal having a bottom portion 1240 for supporting the display assembly 1100 on a ground surface, a top portion 1242 atop which the frame 1200 is supported, and a body portion 1244 extending generally vertically between the bottom and top portions 1240, 1242. In the example illustrated, the bottom portion 1240 comprises a generally horizontally oriented floor plate. The top portion 1242 comprises a generally horizontally oriented mounting plate and defines the top surface 1220 of the base 1198.

Referring to FIG. 11, in the example illustrated, the body portion 1244 has an interior 1245 open to a top end of the body portion 1244, and the top portion 1242 of the pedestal is detachably mounted over the body portion 1244 and covers the top end of the body portion 1244. Referring to FIG. 10, the body portion 1244 has a sidewall with an access opening 1246 to the interior to provide access to, for example, the mounting hardware (e.g. fastener portions) in the interior for attaching and detaching the frame portions 1201, 1202 to the top portion 1242, and the top portion 1242 to the body portion 1244 of the pedestal. An access door 1248 is mounted over the access opening 1246 and is movable between open and closed positions for uncovering and covering the access opening 1246, respectively. In the example illustrated, the access door 1248 has a locking mechanism 1250 movable between a locked position for locking the door 1248 in the closed position, and an unlocked position to permit movement of the door 1248 between the open and closed positions. The locking mechanism 1250 can be key operated to prevent unauthorized access to the interior of the pedestal.

Referring to FIG. 12, in the example illustrated, the display assembly 1100 has a lighting system 1210 for illuminating portions of the display assembly 1100. In the example illustrated, the lighting system 1210 includes at least one first light source 1212 directed toward the display container 1116 for illumination of the contents in the display container 1116 (e.g. the botanical specimen and kaleidoscopic objects). In the example illustrated, the light source 1212 comprises a plurality of light emitting diodes (LEDs) adjacent and directed toward the display container 1116. In the example illustrated, the LEDs are mounted to a light shield 1252 projecting upwardly from the top surface 1220 of the pedestal and positioned generally between the display container 1116 and the kaleidoscope tube 1104. The LEDs are mounted to a front of the light shield 1252 directed toward the display container 1116 for illumination thereof. The light shield 1252 blocks light emitted from the LEDs in the rearward and upward directions toward the kaleidoscope tube 1104.

Referring to FIG. 11, in the example illustrated, each upright member 1204 is of integral, unitary, one-piece construction, and is made of an optical polymer (e.g. Polymethylmethacrylate (Acrylic)) capable of light transmission for illumination thereof. Referring to FIG. 12, in the example illustrated, the lighting system 1210 includes a plurality of second light sources 1260 for emitting light into the upright members 1204 (e.g. for illumination of edges, etched or engraved designs, and/or other etched and/or diffuse surfaces of the upright members). In the example illustrated, the second light sources 1260 comprise a plurality of LEDs positioned on a floor of the recesses 1222 for emitting light upwardly into lower edges of corresponding mounting portions 1224 positioned in the recesses 1222 to illuminate the upright members 1204.

The lighting system 1210 optionally includes power and/or charging ports for connection of the lighting system 1210 to a power source for powering the lighting system 1210 and/or charging a power storage unit (e.g. one or more batteries) for powering the lighting system 1210. The lighting system 1210 optionally includes one or more switches for selectively activating, deactivating, and/or adjusting intensity (e.g. dimming) of the light sources, and one or more indicator lights for indicating, for example, power and/or charging status. Components of the lighting system 1210 (e.g. wiring, power, and/or control components, etc.) can be housed in the interior of the pedestal.

1. A kaleidoscopic display assembly, comprising:
a) a stand;
b) a kaleidoscope tube supported by the stand and extending along a tube axis between a first end and a second end, the tube having an interior extending along the tube axis between a first opening at the first end of the tube and a second opening at the second end of the tube, and a plurality of reflective surfaces arranged in the interior about the tube axis for forming kaleidoscopic patterns;
c) a display container at the second end of the tube, the display container enclosing a first display chamber extending across the second opening of the tube for containing at least a first botanical specimen viewable in a kaleidoscopic pattern through the first opening of the tube; and
d) an aroma container alongside the tube and enclosing an aroma chamber for containing a second botanical specimen, the aroma container having one or more scent ports extending therethrough for sampling an aroma of the second botanical specimen during viewing of the kaleidoscopic pattern through the first opening.

2. The display assembly of claim 1, wherein the aroma container includes an air pump operable to discharge scented air having an aroma of the second botanical sample from the aroma chamber through the one or more scent ports.

3. The display assembly of claim 2, wherein the air pump comprises a resiliently deformable bulb projecting outwardly from a wall of the aroma container, and depressible inwardly toward the aroma chamber to urge discharge of the scented air.

4. The display assembly of claim 1, wherein the display container includes a second display chamber extending across the second opening, the second display chamber containing a plurality of objects for forming part of the kaleidoscopic pattern viewable through the first opening.

5. The display assembly of claim 1, wherein the display container is rotatable relative to the kaleidoscope tube about a display container axis extending parallel with the tube axis for moving the first botanical specimen relative to the reflective surfaces.

6. The display assembly of claim 1, wherein the display container includes a container body and a removable lid attached to the container body, the container body and the lid enclosing the first display chamber.

7. The display assembly of claim 6, wherein the lid comprises a second display chamber containing a plurality of objects for forming part of the kaleidoscopic pattern viewable through the first opening.

8. The display assembly of claim 1, wherein the stand includes a base and a frame extending upwardly from the base, and wherein the kaleidoscope tube, aroma container, and display container are coupled to the frame.

9. The display assembly of claim 1, further comprising a lighting system including at least one light source coupled to the stand for illuminating contents of the display container.

10. A kaleidoscopic display assembly, comprising:
a) a stand;
b) a kaleidoscope tube supported by the stand;
c) a display container in alignment with the kaleidoscope tube for containing at least a first botanical specimen viewable in a kaleidoscopic pattern through a viewing opening of the kaleidoscope tube; and
d) an aroma container adjacent the tube and enclosing an aroma chamber for containing a second botanical specimen, the aroma container having one or more scent ports extending therethrough for sampling an aroma of the second botanical specimen during viewing of the kaleidoscopic pattern.

11. The display assembly of claim 10, wherein the aroma container includes an air pump operable to discharge scented air having an aroma of the second botanical sample from the aroma chamber through the one or more scent ports.

12. The display assembly of claim 10, wherein the display container includes a first display chamber for containing the first botanical specimen and a second display chamber in isolation from the first display chamber and containing a plurality of objects for forming part of the kaleidoscopic pattern.

13. A kaleidoscopic display assembly, comprising:
a) a stand;
b) a kaleidoscope tube supported by the stand; and
c) one or more containers enclosing one or more chambers for containing botanical specimens, wherein one of the chambers is in alignment with the kaleidoscope tube for forming a kaleidoscopic pattern of a corresponding botanical specimen, the kaleidoscopic pattern viewable through a viewing opening of the kaleidoscope tube, and wherein one of the containers includes one or more scent ports for sampling an aroma of a corresponding botanical specimen during viewing of the kaleidoscopic pattern through the viewing opening.

14. The display assembly of claim 13, wherein the one of the containers includes an air pump operable to discharge scented air having an aroma of the corresponding botanical specimen through the one or more scent ports.

15. A method of displaying botanical specimens for multisensory sampling, comprising:
a) inserting a first botanical specimen into a display chamber mounted in alignment with a kaleidoscope tube for forming a kaleidoscopic pattern of the first botanical specimen viewable through the kaleidoscope tube; and
b) inserting a second botanical specimen into an aroma container mounted adjacent the kaleidoscope tube, the aroma container having one or more scent ports for sampling an aroma of the second botanical specimen during viewing of the kaleidoscopic pattern.

16. The method of claim 15, wherein the first botanical specimen and the second botanical specimen are of a common strain.

17. A kaleidoscopic display assembly, comprising:
a) a stand;
b) a kaleidoscope tube supported by the stand, the tube having an interior extending along a tube axis between a first opening and a second opening opposite the first opening, and a plurality of reflective surfaces arranged in the interior about the tube axis for forming kaleidoscopic patterns;
c) a first display chamber extending across the second opening for containing a botanical specimen viewable in a kaleidoscopic pattern through the first opening; and
d) a second display chamber extending across the second opening between the first display chamber and the second opening of the kaleidoscope tube, the second display chamber containing a plurality of objects for forming part of the kaleidoscopic pattern viewable through the first opening.

18. The display assembly of claim 17, wherein the first display chamber and the second display chamber are in a common display container coupled to the stand.

19. The display assembly of claim 18, wherein the display container comprises a container body and a lid removably attached to the container body, the container body and the lid bounding the first display chamber, and wherein the second chamber is internal the lid.

20. The display assembly of claim 17, wherein each of the first and second display chambers are rotatable relative to the kaleidoscope tube for moving the botanical specimen and the plurality of objects relative to the reflective surfaces.
